SEQUENCE LISTING (4 sequences)

SEQ ID NO: 1 - DNA, 18 bp, Artificial Sequence
  G72 forward primer
gagcattcttctgagcaa

SEQ ID NO: 2 - DNA, 19 bp, Artificial Sequence
  G72 reverse primer
ccttctcttccatccttct

SEQ ID NO: 3 - DNA, 970 bp, Homo sapiens
tgctgaatggaaagccagaaagtagagtgaagcaagtaatgtgtgtgtgagtagtcattggatacatacataacagtgagcaagtttatcagctcctgcatggcagtgttctgatgatcttttgctgcaaaagagctacaccccaaattagtggcttaaagtaaaagcttactagtgtatatgatgattctgttggtccaggatttggaaagggcatggcaggaggtctcatctctgcttcacaatgccgatgatttagctgggaggacccaaaatgctggaaaagctgatgggtgctgattctctccagcttttcagatccagatatacattgggtaaaatctacttcataggttttcaaaggagcattcttctgagcaaatctgaaaactctctaaactctattgcaaaggagacagaagaaggaagagagacggtaacaaggaaagaaggatggaagagaaggcatgaggacggctatttggaaatggcacagaggcatttacagagatcattatgtccttgggtctcttaccttcctcagccctatgcagagcttgaagaagtaagcagccatgttggaaaagtcttcatggcaagaaactatgagttccttgcctatgaggcctctaaggaccgcaggcagcctctagaacgaatgtggacctgcaactacaaccagcaaaaagaccagtcatgcaaccacaaggaaataacttctaccaaagctgaatgagtttggaagcagattcttcccagccaatccttctgatgacaatgtagtctggccaacatcttcactggactctgacggactctgtgtctgggacccagctgataacacgtggtgatgggattgtatttgcaactctctggtcagtaagtgataaaatgccatttctatgcacccacctggcctgtgtgactgggagaatctctctttttattaaatgtgcttcaagttttaaca

SEQ ID NO: 4 - protein, 153 aa, Homo sapiens
MLEKLMGADSLQLFRSRYTLGKIYFIGFQRSILLSKSENSLNSIAKETEEGRETVTRKEGWKRRHEDGYLEMAQRHLQRSLCPWVSYLPQPYAELEEVSSHVGKVFMARNYEFLAYEASKDRRQPLERMWTCNYNQQKDQSCNHKEITSTKAE

(19) United States
(12) Patent Application Publication
(10) Pub. No.: US 2014/0288055 A1
(43) Pub. Date: Sep. 25, 2014

(54) COMPOSITIONS AND METHODS FOR DIAGNOSIS OF SCHIZOPHRENIA

(71) Applicant: China Medical University, Taichung (TW)

(72) Inventors: Hsien-Yuan Lane, Taichung (TW); Guochuan Emil Tsai, Taichung (TW); Hao-Teng Chang, Taichung (TW); Chieh-Hsin Lin, Taichung (TW)

(73) Assignee: China Medical University, Taichung (TW)

(21) Appl. No.: 14/248,742

(22) Filed: Apr. 9, 2014

Related U.S. Application Data
(63) Continuation-in-part of application No. 13/713,102, filed on Dec. 13, 2012, now abandoned.

(51) Int. Cl. G01N 33/68; C12Q 1/68

(52) U.S. Cl. CPC G01N 33/6893; C12Q 1/6883
USPC 514/211.13; 436/501; 435/7.92; 435/6.11

(57) ABSTRACT

Provided is a method for diagnosis of schizophrenia, which comprises: detecting G72 gene product in a body fluid sample from a subject by an assay to determine G72 expression level; comparing said G72 expression level to a baseline G72 expression; and relating the G72 expression level to the patient's risk of schizophrenia by assigning an increased risk of schizophrenia when said G72 expression level is greater than said baseline G72 expression. Through detecting G72 expression level in a peripheral sample, the method can be simply performed by an in vitro assay and accurately predict or diagnose a subject with schizophrenia.

### RELATED PATENT APPLICATION

This application is a continuation-in-part application and claims priority from subject matter disclosed in the earlier filed patent application Ser. No. 13/713,102, filed on Dec. 13, 2012, which is incorporated herein in its entirety by reference.

### BACKGROUND OF THE INVENTION

1. Field of the Invention

The invention relates a method for diagnosis of schizophrenia, particularly to a method for evaluating risk of a subject having, developing, or going to have schizophrenia by determining peripheral G72 expression.

2. Description of the Prior Arts

Evidences from the views of neurodevelopment, pathological finding, genetic vulnerability, animal models, pharmacology and clinical trials provide supports that glutamatergic signaling, particularly the molecules of the NMDA synapse, plays an important role in the pathophysiology of schizophrenia (Harrison P J, Weinberger D R. Mol Psychiatry 2005; 10(1): 40-68; image 45; Lin C H, Lane H Y, Tsai G E. Pharmacol Biochem Behav 2012; 100(4): 665-677). In clinical trials, NMDA-enhancing agents resulted in a significant reduction of symptomatology in patients with schizophrenia (Lane H Y, Chang Y C, Liu Y C, Chiu C C, Tsai G E. Arch Gen Psychiatry 2005; 62(11): 1196-1204).

The gene encoding G72 protein was confirmed a schizophrenia susceptibility gene over certain ethnic populations (Harrison P J et al. supra; Wang X, He G, Gu N, Yang J, Tang J, Chen Q et al. Biochem Biophys Res Commun 2004; 319(4): 1281-1286). G72 protein is preferentially expressed in brain regions associated with schizophrenia (Korostishevsky M, Kaganovich M, Cholostoy A, Ashkenazi M, Ratner Y, Dahary D et al. Biol Psychiatry 2004; 56(3): 169-176). Moreover, inconsistency of predicted susceptibility of schizophrenia via G72 genotyping, such as linkage disequilibrium analysis and haplotype analysis, results in failure of providing a reliable method for diagnosis of schizophrenia for all populations, particularly for Asian population. Although there is an increase of G72 transcripts in the dorsolateral prefrontal cortex of schizophrenia patients (Korostishevsky et al., 2004; supra), the current techniques still fail to provide a precise and consistent method for predicting susceptibility of a subject to schizophrenia in a determined collected diagnostic sample.

Further, individuals with overactive G72 protein were found to exhibit attenuated NMDA activity by lowering the co-agonists, predisposing them to schizophrenia (Boks M P, Rietkerk T, van de Beek M H, Sommer I E, de Koning T J, Kahn R S. Eur Neuropsychopharmacol 2007; 17(9): 567-572). However, it is completely silent to the relationship of peripheral G72 expression with schizophrenia and none of the current art has revealed that determination of peripheral expression of G72 is an applicable technical means for diagnosis of schizophrenia.

To overcome the shortcomings, the present invention provides a method for diagnosis of schizophrenia via a simplified technical means with a reliable effectiveness to mitigate or obviate the aforementioned problems.

### SUMMARY OF THE INVENTION

The main objective of the present invention is to provide a reliable diagnostic method for predicting risk of schizophrenia in a subject, which is applicable for all populations, particularly to Asian population.

Accordingly, in one aspect, the present invention provides a method for diagnosis of schizophrenia, which comprises: detecting G72 gene product in a body fluid sample by an assay to determine G72 expression level; comparing said G72 expression level to a baseline G72 expression; and relating the G72 expression level to the subject's risk of schizophrenia by assigning an increased risk of schizophrenia when said G72 expression level is greater than said baseline G72 expression.

According to the present invention, the term “G72 expression level” hereby refers to the degree of expression of G72 gene product in post-transcriptional stage or post-translational stage. Particularly, G72 expression level reflects the amount of protein or mRNA or protein encoded by G72 gene in a diagnostic sample from a subject.

According to the present invention, the baseline G72 expression is determined by a reference sample from a normal subject. Preferably, the reference sample is blood, blood cells, plasma, serum, urine, or saliva from the normal subject. Said normal subject is a human subject who has no schizophrenia or is unlikely to develop schizophrenia or a condition relating thereto. Preferably, the age, gender of the normal subject is selected to correspond to the subject pursuing diagnosis of schizophrenia.

The present invention also provides a method of diagnosing schizophrenia, which comprises: detecting from a sample obtained from the human subject which comprises nucleic acids which encodes G72 protein or its fragment, or detecting from the sample G72 protein or its fragment; and determining schizophrenia in the human subject based on the increased expression of G72 in peripheral. Preferably, said nucleic acids comprise a fragment of the sequence as set forth in SEQ ID NO: 3; and G72 protein or its fragment comprises a fragment of the sequence as set forth in SEQ ID NO: 4. In accordance with the present invention, a nucleic acid region is amplified by a primer pair of SEQ ID NO: 1 and SEQ ID NO: 2; or any primer pair to amplify a fragment of G72 gene with a sequence as set forth in SEQ ID NO: 3, particularly a partial sequence as set forth in SEQ ID NO: 3, wherein said G72 gene product is a biomarker for schizophrenia.

In a second aspect, the present invention also provides a method for determination of treatment regimens in subjects of schizophrenia, which includes:

performing an assay that detects G72 gene product in a body fluid sample to determine G72 expression level; comparing said G72 expression level to a baseline G72 expression; and relating the G72 expression level to the subject's risk of schizophrenia by assigning an increased risk of schizophrenia when said G72 expression level is greater than said baseline G72 expression; and administrating a drug for ameliorating or alleviating symptoms of schizophrenia. According to the present invention, the drug could be but not limited to

risperidone, zotepine, haloperidol, quetiapine, amisulpride, sulpiride, flupentixol, olanzapine, ziprasidone, chlorpromazine, paliperidone and any other drug for treating schizophrenia.

In a third aspect, the present invention also provides a composition or kit for diagnosis of schizophrenia, which comprises: a G72 probe recognizing the G72 gene product by specifically binding thereto, which is adapted to detect expression of G72 gene product. In accordance with the present invention, the G72 probe as known in the art could be any of molecules recognizing the G72 protein, G72 mRNA or G72 cDNA; for example, Anti-G72 antibody, G72 affibody, molecules containing complementarity-determining region (CDR) which recognizes the epitope of G72, and G72 gene sequence fragment, such as G72 primers designed according to the sequence of G72 cDNA sequence.

The present invention provides a method for diagnosis of schizophrenia based on that patients with schizophrenia have a remarkable elevation of G72 protein expression in plasma when compared to healthy controls. The method is proved to be consistent and applicable by showing an increase in G72 transcripts in the postmortem brains from patients with schizophrenia supports that G72 is a susceptibility protein to schizophrenia. The present invention indicates that the peripheral expression of a single G72 protein is a diagnostic biomarker for schizophrenia with favorable sensitivity and specificity. Through detecting G72 expression level in a peripheral sample, the method in accordance with the present invention can be simply performed by an in vitro assay, which can avoid use of invasive means to obtain biopsies or fluid around brain region.

Other objectives, advantages and novel features of the invention will become more apparent from the following detailed description when taken in conjunction with the accompanying drawings.

### BRIEF DESCRIPTION OF THE DRAWINGS

FIG. 1 demonstrates typical western blot results for G72 protein in plasma samples from randomly selected medicated schizophrenia patients (n=8), drug-free schizophrenia patients (n=8) and healthy controls (n=8);

FIGS. 2A, 2B, 2C and 2D respectively illustrate results of ROC curve analysis of plasma G72 protein levels of healthy controls vs. schizophrenia patients, wherein FIG. 2A refers to healthy controls (I) vs. unmatched medicated schizophrenia; FIG. 2B refers to healthy controls (I) vs. matched medicated schizophrenia; FIG. 2C refers to healthy controls (II) vs. unmatched drug-free schizophrenia; and FIG. 2D refers to healthy controls (II) vs. matched drug-free schizophrenia patients;

FIGS. 3A and 3B respectively illustrate expression levels of G72 protein in plasma of healthy controls (I) vs. medicated schizophrenia patients (FIG. 3A); and healthy controls (II) vs. drug-free schizophrenia patients (FIG. 3B), wherein *** represents P<0.0001;

FIG. 4A and FIG. 4B respectively illustrate expression levels of G72 mRNA and endogenous control (GAPDH) mRNA in leukocytes. Four lines are rendered background fluorescence signal disturbance, no G72 specificity PCR product peak was shown (FIG. 4A). The same four samples to detect the endogenous gene (GAPDH) expression in leukocytes results (as an internal control group) in FIG. 4B. The four lines appear near 80° C. are specificity PCR product single peak, so that leukocytes have normal housekeeping gene expression, but no G72 mRNA expression.

### DETAILED DESCRIPTION OF THE PREFERRED EMBODIMENTS

Provided is a method for diagnosis of schizophrenia, which comprises: detecting G72 gene product in a body fluid sample from a subject by an assay to determine a G72 expression level; comparing said G72 expression level to a baseline G72 expression; and relating the G72 expression level to the subject's risk of schizophrenia by assigning an increased risk of schizophrenia when said G72 expression level is greater than said baseline G72 expression.

According to the present invention, the step of detecting G72 in a body fluid sample from a subject by an assay to determine G72 expression level includes detecting G72 expression level by any detection method as known in the art of proteomics or molecular biology, such as western blotting, enzyme-linked immunosorbent assay (ELISA), northern blotting, polymerase chain reaction (PCR), reverse transcriptase-polymerase chain reaction (RT-PCR) analysis, realtime-polymerase chain reaction, and chemiluminescent Immunoassay.

In the method for diagnosis of schizophrenia according to the present invention, the body fluid sample is blood, blood cells, plasma, serum, urine, or saliva. Alternatively, the body fluid sample is processed from blood, blood cells, plasma, serum, urine, or saliva by eliminating the undesired portions; for example, by depleting fat or other substances containing no genetic information or G72 gene product.

As known in the art, G72 protein may play an important role in the modulation of NMDA signaling. In vitro study indicates that G72 protein activates DAAO (Chumakov I, Blumenfeld M, Guerassimenko O, Cavarec L, Palicio M, Abderrahim H et al. Proc Natl Acad Sci USA 2002; 99(21): 13675-13680), which oxidizes D-amino acids such as D-serine and D-alanine, both of which are co-agonists at the glycine site of the NMDA receptor. G72 is also named as D-amino acid oxidase activator (DAOA) (Genbank Accession No. NM_{—}172370). Any other known isomers of G72 protein with a minor modification or polymorphism can also be the target as G72 protein to detect in the method for diagnosis of schizophrenia in accordance with the present invention.

Provided herein are methods for determining whether a subject has schizophrenia or is likely to develop schizophrenia or a condition relating thereto. As used herein, “schizophrenia” refers to all types and stages of schizophrenia, including, but not limited to: first onset, acute episode, subacute phase, chronic phase, prodromal phase and high risk individuals. Early stages of schizophrenia include “conditions relating to schizophrenia”, e.g., hallucinations, delusions, disorganized speech, disorganized or catatonic behavior, negative symptoms (such as alogia, avolition and so on), and any other defficiecy of cognition. The methods described herein may also predict the presence or likelihood of development of the early stages of schizophrenia and conditions relating to schizophrenia. For example, the methods described herein may determine: the likelihood of a symptomless subject developing schizophrenia, an early stage of schizophrenia, or a condition relating thereto; the likelihood of a subject having symptoms, e.g., symptoms that resemble those present in early stage schizophrenia, to have or to develop schizophrenia or an early stage thereof or a condition relating thereto; the likelihood of a subject having early stage schizophrenia symptoms to develop schizophrenia; or the likelihood of a subject having early stage schizophrenia symptoms to develop any phase of schizophrenia.

The method may be used to determine whether a subject is more likely than not to have schizophrenia, an early stage thereof, or a condition relating thereto, or is more likely to have schizophrenia, an early stage thereof, or a condition relating thereto than to have another disease, based on the difference between the measured and standard level or reference range of G72 gene product.

Thus, for example, a patient with a putative diagnosis of schizophrenia or a condition relating thereto may be diagnosed as being “more likely” or “less likely” to have schizophrenia in light of the information provided by a method described herein. If a plurality of biomarkers are measured for diagnosis of schizophrenia, at least one and up to all of the measured biomarkers must be G72 gene product, in the appropriate direction, for the subject to be diagnosed as having (or being more likely to have) schizophrenia or a condition relating thereto. The methods described herein are not limited to making an initial diagnosis of schizophrenia and a condition relating thereto, but are also applicable to confirming a provisional diagnosis of schizophrenia or a condition relating thereto, or for “ruling out” such a diagnosis.

For the assay that detects G72 gene product in the body fluid sample, the used G72 probe can be polynucleotides labeled with a signal-producing agent, which emits signal for detecting the presence of or quantifying the amount of the target. As known in the art, signal-producing agent could be fluorescence dye or isotopic element. The kit in accordance with the present invention further comprises a reference sample from a normal subject. Said normal subject is a human subject who has no schizophrenia or is unlikely to develop schizophrenia or a condition relating thereto.

In a preferred embodiment in accordance with the present invention, the assay can be, but not limited to: western blotting, Enzyme-linked Immunosorbent Assay (ELISA) or chemiluminescent Immunoassay.

In another preferred embodiment in accordance with the present invention, the assay can be, but not limited to; polymerase chain reaction (PCR), reverse transcriptase-polymerase chain reaction (RT-PCR) analysis and realtime-polymerase chain reaction.

In yet another preferred embodiment in accordance with the present invention, the G72 probe is anti-G72 antibody or a polynucleotide with a sequence having at least ten consecutive nucleotides as set forth in SEQ ID NO. 3. More preferably, the G72 probe includes at least one polynucleotide with the sequence as set forth in SEQ ID NO. 1 or SEQ ID NO. 2.

The present invention indicates that the peripheral expression of a single G72 protein is an applicable diagnostic biomarker for schizophrenia with favorable sensitivity and specificity. Therefore, the folds of G72 expression level with respect to the baseline G72 expression is alternative when the method in accordance with the present invention is subjected to various populations. Once the G72 expression level is greater than said baseline G72 expression, the subject is determined to have an increased risk of developing schizophrenia.

In some embodiments, said G72 expression level is at least 2-fold, 3-, 4-, 5-, 10- or more fold, with respect to greater than said baseline G72 expression.

The invention will be further illustrated by the following non-limiting Examples, with reference to the accompanying Figures, in which:

### EXAMPLE

### 1. Materials and Methods

Participants

All experiments herein were approved by the institutional review board of China Medical University Hospital, Taiwan, and carried out in accordance with the Declaration of Helsinki. Consecutive patients were screened and recruited from the psychiatric treatment programs of China Medical University, which is a major medical center in central Taiwan. The patient population is similar to that of other mental health facilities. After complete description of the study to the subjects, written informed consent was obtained in line with the IRB's guidelines.

The following example includes two independent cohorts of patients and healthy controls: the learning set and the testing set. In the two independent cohorts, both patients and controls were Han Chinese, aged 18-50 years, who were physically and neurologically healthy and had normal laboratory assessments (including urine/blood routine, biochemical tests, and electrocardiograph). Both patients and controls were evaluated by Diagnostic and Statistical Manual of Mental Disorders, Forth Edition for diagnosis. All patients had schizophrenia. Patients with Axis I diagnosis other than schizophrenia, or any Axis II diagnosis were not included. All healthy volunteers were free of any Axis I or II psychiatric disorder. To exclude potential confounding effects, all participants were non-smokers and had no DSM-IV diagnosis of substance (including alcohol) abuse.

For examining possible drug effects on the protein expression, both psychotropic-free (for >3 months) and medicated schizophrenia patients were recruited. Psychotropic status was ascertained by interviewing the patients and family members or care givers, contacting other health care providers and reviewing chart. Healthy controls had no history of exposure to psychotropics. All drug-free schizophrenia patients had not taken any psychotropic medication for more than three months. All drug-free patients with schizophrenia were recruited from the outpatient clinic, and all medicated schizophrenia patients were from the inpatient unit. All the patients continued to receive standard psychiatric care after the initial evaluation and blood draw.

Among 66 schizophrenia patients, 27 were psychotropic-free for 3 months or longer and the other 39 were stabilized on antipsychotics (11 risperidone, 5 zotepine, 5 haloperidol, 4 quetiapine, 4 amisulpride, 3 sulpiride, 3 flupentixol, 1 olanzapine, 1 ziprasidone, 1 chlorpromazine, 1 paliperidone) for at least 3 months.

Determination of G72 Level

To investigate the G72 level in the plasma, the protein expression levels were examined with western blot analysis. Ten mL of blood was collected into EDTA-containing blood collection tubes by personnel trained in phlebotomy using sterile technique. The blood specimens were processed immediately by centrifugation at 500 g. After centrifugation, plasma was quickly dissected, immediately stored at −80° C. until western blotting.

For western blotting, 100 μL plasma was depleted using ProteoPrep® Blue Albumin and IgG Depletion Kit (Sigma). The low-abundant protein fractions were collected to 100 μL. Then, 10 μL of the fractions were mixed with 4× sample buffer (500 mM Tris-HCl (pH 6.8), 16% SDS, 80% glycerol, 400 mM DTT, and 0.08% bromophenol blue) and separated on 12% SDS-PAGE. Proteins in the gels were transferred to 0.45 μm polyvinylidene difluoride (PVDF) membrane (Millipore). Membranes were placed in 5% nonfat dry milk in TBST (20 mM Tris-HCl pH 7.6, 500 mM sodium chloride, 0.1% Tween 20) for 1 hour at room temperature, and then incubated with goat anti-G72 antibody (G72(N15):sc-46118, Santa Cruz Biotechnology) diluted 1:1000 in TBST overnight at 4° C. The membranes were washed for 3 times for 15 minutes in TBST and incubated for 2 hours with a HRP-linked anti-goat IgG secondary antibody (sc-2030, Santa Cruz Biotechnology) diluted in 1:5000 TBST. After 3 washes in TBST, the blots were visualized with an ECL Advance Western Blotting Detection Kit (RPN2135, GE Healthcare). The stained membranes were then be photographed on ImageQuant LAS 4000 mini (GE Healthcare) and quantified using ImageQuant™ TL 7.0 software (GE Healthcare) by measuring the relative intensity from each band and normalized to the G72 recombinant protein (20 or 50 ng) signals (kindly donated by Dr. Hao-Teng Chang, Graduate Institute of Molecular Systems Biomedicine, China Medical University). All western blot analyses were repeated for two times.

Melting Curve Analysis

Melting curve analysis is an assessment of the dissociation-characteristics of PCR product during heating. As the temperature is raised, the double strand begins to dissociate leading to a rise in the absorbance and intensity of hyperchromicity. The temperature at which 50% of DNA is denatured is known as the melting point. The fluorescent variation is in accordance with the sequence or fragment, and the same sequence or fragment would display identical fluorescent variation as well as the only one peak.

Statistical Analysis

All subjects' clinical characteristics and western blot data were presented as mean±SD or number (percentage). All statistical methods were performed using IBM SPSS Statistics version 18.0 (SPSS inc.) and MedCalc statistical software version 11.6 (MedCalc Software). All means between groups were compared using independent t test or Mann-Whitney U test for two groups, one-way ANOVA or Kruskal-Wallis test for two groups and percentages using χ2 test. Stepwise logistic regression and Receiver Operating Characteristics (ROC) analysis (hereby noted as ROC curve analysis) were used to generate predictive models and to evaluate the significant predictors of schizophrenia patients. A P value less than 0.05 was considered statistically significant.

### 2. Results

### 2.1. The Learning Set:

The participants of the learning set were 30 unrelated healthy controls (healthy controls I) and 39 medicated schizophrenia patients. There was no significant difference in gender distribution between the unmatched healthy controls I and medicated schizophrenia patients. The mean age of unmatched medicated schizophrenia patients (38.8±9.1, p=0.011) was older than that of healthy controls I (33.0±9.1). Thirty patients from the medicated schizophrenia group were selected to match with healthy controls I by age and gender. The demographic data of age-, gender-matched and unmatched medicated schizophrenia patients and healthy controls I are summarized in Table 1.

TABLE 1
Demographic characteristics of medicated schizophrenia patients and healthy controls (I)

| | Unmatched | | | Matched | | | |
|---|---|---|---|---|---|---|---|
| | Healthy | Medicated | | Healthy | Medicated | | |
| Parameter | controls (I) | schizophrenia | P-value | controls (I) | schizophrenia | P-value | P-value |
| N | 30 | 39 | | 30 | 30 | | |
| Gender | | | 0.603 | | | 0.580 | |
| Male | 19 (63.3%) | 28 (71.8%) | | 19 (63.3%) | 22 (73.3%) | | |
| Female | 11 (36.7%) | 11 (28.2%) | | 11 (36.7%) | 8 (26.7%) | | |
| Age (year) | 33.0 ± 9.1 | 38.8 ± 9.1 | 0.011 | 33.0 ± 9.1 | 35.9 ± 7.8 | 0.188 | |
| Education (year) | | 11.0 ± 1.7 | | | 11.0 ± 1.7 | | 0.950 |
| Age at onset (year) | | 22.3 ± 6.1 | | | 20.8 ± 4.5 | | 0.259 |
| Illness duration (m) | | 191.9 ± 91.2 | | | 177.1 ± 87.5 | | 0.500 |
| PANSS total score | | 87.6 ± 12.3 | | | 88.1 ± 12.7 | | 0.872 |
| G72 level (ng/μL) | 1.17 ± 0.57 | 4.43 ± 2.84 | <0.0001 | 1.17 ± 0.57 | 4.60 ± 3.09 | <0.0001 | |

There was no significant difference between the matched healthy controls I and medicated schizophrenia patients in age and gender (p=0.188 and 0.580, respectively). The mean educational year, age at onset, illness duration and PANSS total score were not different between unmatched and matched medicated schizophrenia patients (P>0.05) (Table 1).

The Plasma G72 Protein Expression was Higher in Medicated Schizophrenia Patients than in Healthy Controls I.

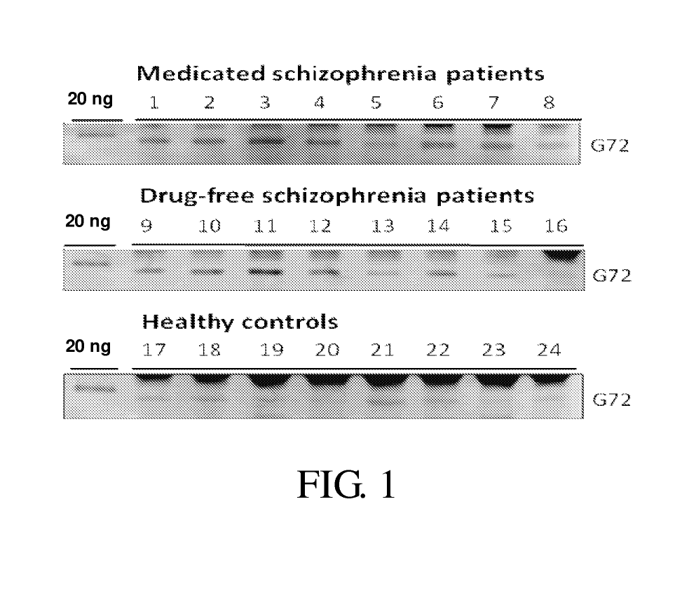

As shown in FIG. 1, the expression levels of G72 in the plasma of medicated schizophrenia patients were markedly higher than that of healthy controls I. The mean expression levels (SD) of G72 protein in unmatched medicated, matched medicated schizophrenia patients, and healthy controls I were 4.43±2.84 ng/μL, 4.60±3.09 ng/μL, 1.17±0.57 ng/μL, respectively (Table 1). In multivariate logistic regression analyses, plasma G72 protein expression level was significantly associated with both unmatched medicated schizophrenia (OR=110.08, 95% CI=5.47-2217.68, P=0.002) and matched medicated schizophrenia (OR=92.69, 95% CI=4.69-1831.82, P=0.003) after adjustment for age and gender (data not shown). The G72 protein level was not correlated with illness duration of the medicated schizophrenia patients (r²=−0.038, P=0.844) (data not shown).

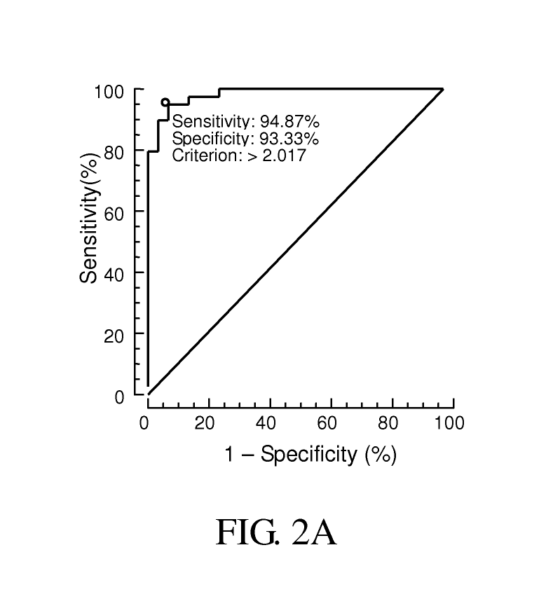

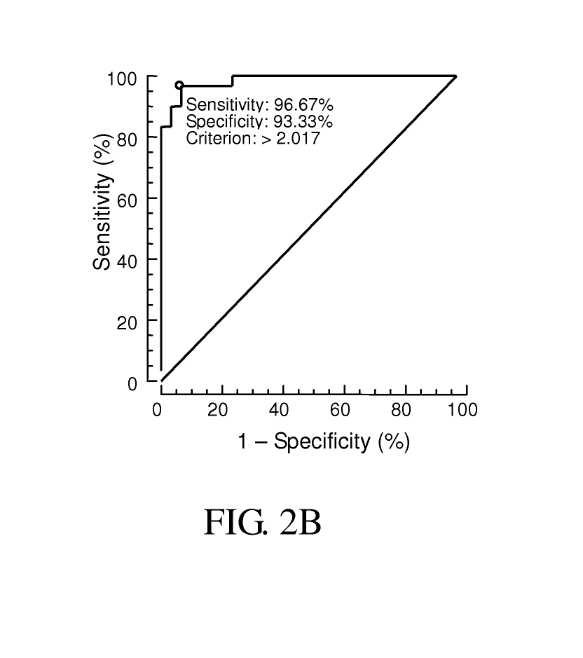

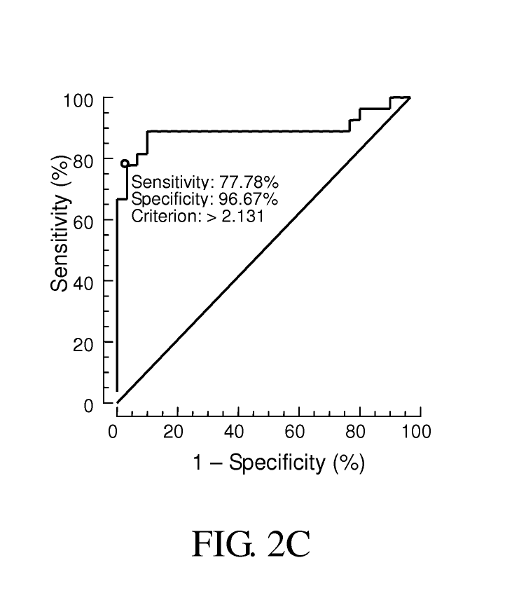

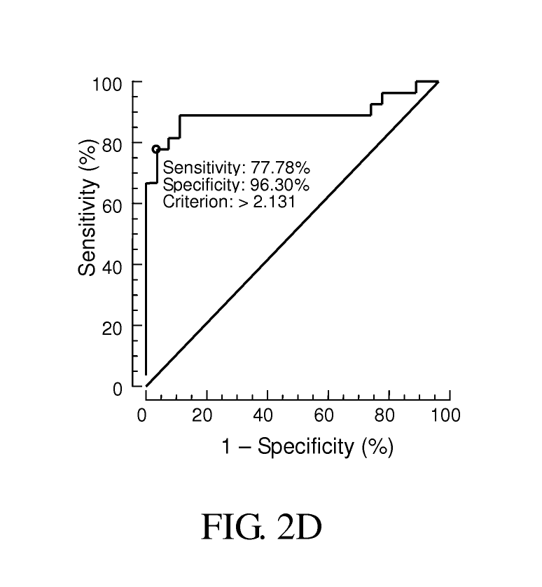

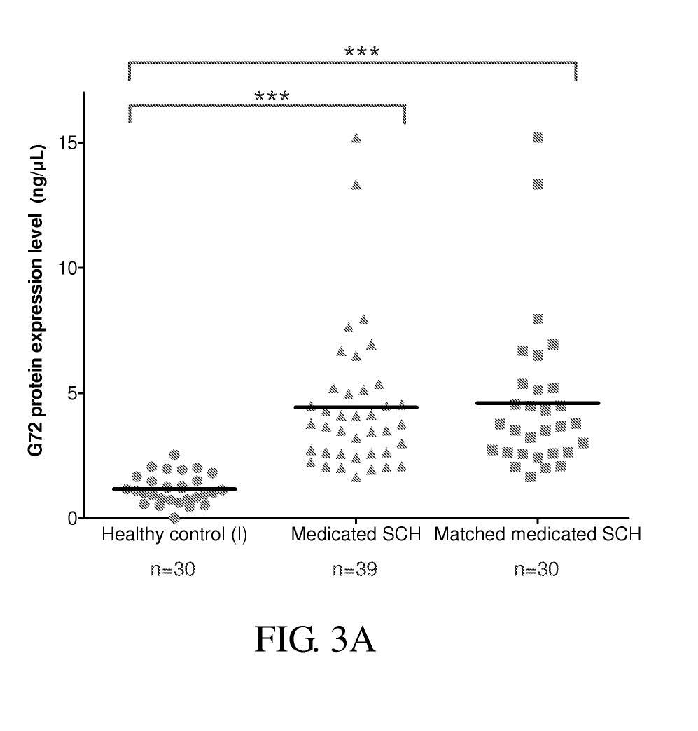

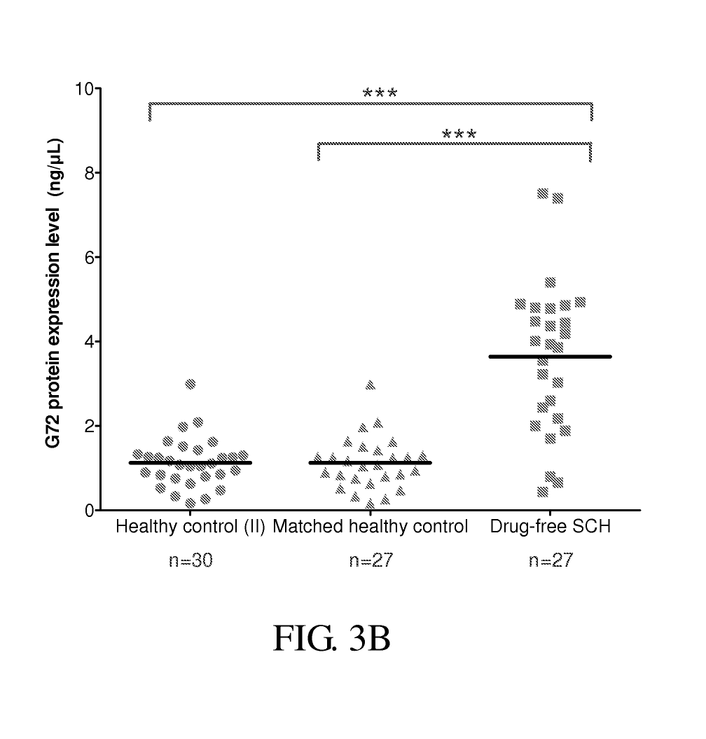

ROC curve analysis was applied to determine the cutoff value of plasma G72 protein expression as the diagnostic predictor for schizophrenia by plotting the proportion of true-positive results (sensitivity) vs. the proportion of false-positive results (1−specificity). The ROC curve analysis for the unmatched medicated schizophrenia patients vs. healthy controls I determined an optimal cutoff value, 2.017, with an excellent sensitivity (94.9%) and specificity (93.3%) (AUC=0.984) (Table 3 and FIG. 2A). When the medicated schizophrenia patients were matched with healthy controls I by age and gender, the optimal cutoff value was also 2.017, providing a sensitivity of 0.967 and specificity of 0.933 (AUC=0.986) (Table 2 and FIG. 2B).

TABLE 2
ROC curve analysis and multivariate logistic regression of plasma
G72 protein level of healthy controls vs. schizophrenia patients

| | | | | | Logistic regression* | |
|---|---|---|---|---|---|---|
| | ROC curve analysis | | | | | Correct |
| | | Sensi- | Speci- | | | Classi- |
| | | tivity | ficity | | | fication |
| | Cut-off | (%) | (%) | AUC | β (SE) | (%) |
| Unmatched | >2.017 | 94.9 | 93.3 | 0.984 | 5.85 | 94.1 |
| medicated SCH | | | | | (1.24) | |
| Matched | >2.017 | 96.7 | 93.3 | 0.986 | 6.08 | 95.0 |
| medicated SCH | | | | | (1.32) | |
| Unmatched | >2.131 | 77.8 | 96.7 | 0.895 | 4.63 | 87.7 |
| drug-free SCH | | | | | (1.14) | |
| Matched | >2.131 | 77.8 | 96.3 | 0.896 | 4.53 | 87.0 |
| drug-free SCH | | | | | (1.14) | |

*Adjusted with age and gender
SCH: schizophrenia

### 2.2. The Testing Set:

The participants of the testing set were another 30 unrelated healthy controls (healthy controls II) and 27 drug-free schizophrenia patients. There was no significant difference between the healthy controls II and drug-free schizophrenia patients in age and gender (P>0.05). Twenty-seven individuals from the healthy control II group were selected to match with drug-free schizophrenia patients better on age and gender. The demographic data of age-, gender-matched and unmatched drug-free schizophrenia patients and healthy controls II are summarized in Table 3. There was no significant difference between the matched healthy controls II and drug-free schizophrenia patients on age and gender (P=0.978 and 0.412, respectively).

TABLE 3
Demographic characteristics of drug-free schizophrenia patients and healthy controls (II).

| | Unmatched | | | Matched | | |
|---|---|---|---|---|---|---|
| | Healthy | Drug-free | | Healthy | Drug-free | |
| Parameter | controls (II) | schizophrenia | P-value | controls (II) | schizophrenia | P-value |
| N | 30 | 27 | | 27 | 27 | |
| Gender | | | 0.186 | | | 0.412 |
| Male | 17 (56.7%) | 10 (37.0%) | | 14 (51.9%) | 10 (37.0%) | |
| Female | 13 (43.3%) | 17 (63.0%) | | 13 (48.1%) | 17 (63.0%) | |
| Age (year) | 32.6 ± 10.9 | 31.7 ± 9.1 | 0.740 | 31.7 ± 10.6 | 31.7 ± 9.1 | 0.978 |
| Education (year) | | 12.6 ± 1.9 | | | 12.6 ± 1.9 | |
| Age at onset (year) | | 25.3 ± 7.7 | | | 25.3 ± 7.7 | |
| Illness duration (m) | | 75.0 ± 66.3 | | | 75.0 ± 66.3 | |
| PANSS total score | | 108.3 ± 21.5 | | | 108.3 ± 21.5 | |
| G72 level (ng/μL) | 1.13 ± 0.58 | 3.64 ± 1.80 | <0.0001 | 1.12 ± 0.61 | 3.64 ± 1.80 | <0.0001 |

The Plasma G72 Protein Expression was Higher in Drug-Free Schizophrenia Patients than in Healthy Controls II.

The expression levels of G72 in the plasma of drug-free schizophrenia patients were higher than that of healthy controls II (FIG. 1). The mean expression levels (SD) of G72 protein in unmatched healthy controls II, matched healthy controls II, and drug-free schizophrenia patients were 1.13±0.58 ng/μL, 1.12±0.61 ng/μL, 3.64±1.80 ng/μL, respectively (Table 3). In multivariate logistic regression analyses, plasma G72 protein expression level was significantly associated with both unmatched drug-free schizophrenia (OR=5.40, 95% CI=2.20-13.24, P<0.001) and matched drug-free schizophrenia (OR=5.15, 95% CI=2.12-12.50, P<0.001) after adjusting the effect of age and gender (data not shown). The G72 protein level was not correlated with illness duration of the drug-free schizophrenia patients (r²=0.074, P=0.715) (data not shown).

The ROC curve analysis for the unmatched drug-free schizophrenia patients vs. healthy controls II determined an optimal cutoff value, 2.131, with an excellent sensitivity (77.8%) and specificity (96.7%) (AUC=0.895) (Table 2 and FIG. 2C). For matched drug-free schizophrenia patients vs. healthy controls II, the optimal cutoff value was also 2.131, providing a sensitivity of 77.8% and specificity of 96.3% (AUC=0.896) (Table 2 and FIG. 2D).

The previous example proved that peripheral G72 protein expression may be a useful surrogate for G72 protein expression in the CNS. To examine whether G72 is over-expressed in patients with schizophrenia, G72 protein levels in peripheral plasma in unrelated patients with schizophrenia and healthy controls were measured. The results of the example suggest that patients with schizophrenia have a remarkable elevation of G72 protein expression in plasma when compared to healthy controls. The finding which is consistent with the study showing an increase in G72 transcripts in the postmortem brains from patients with schizophrenia supports that G72 is a susceptibility protein. To our knowledge, the present invention is the first one to indicate that the peripheral expression of a single protein, particularly to G72 protein, is an applicable diagnostic biomarker for schizophrenia with favorable sensitivity and specificity.

2.3 mRNA Result

The objective of the embodiments is to measure G72 mRNA level from subjects by reverse transcriptase PCR, and to diagnosis of schizophrenia in vitro; wherein the G72 probe is a polynucleotide with a sequence having at least ten consecutive nucleotides as set forth in SEQ ID NO. 3. More preferably, the G72 probe includes at least one polynucleotide with the sequence as set forth in SEQ ID NO. 1 or SEQ ID NO. 2, and the G72 gene product is a blood sample (white blood cell, WBC) from different subjects.

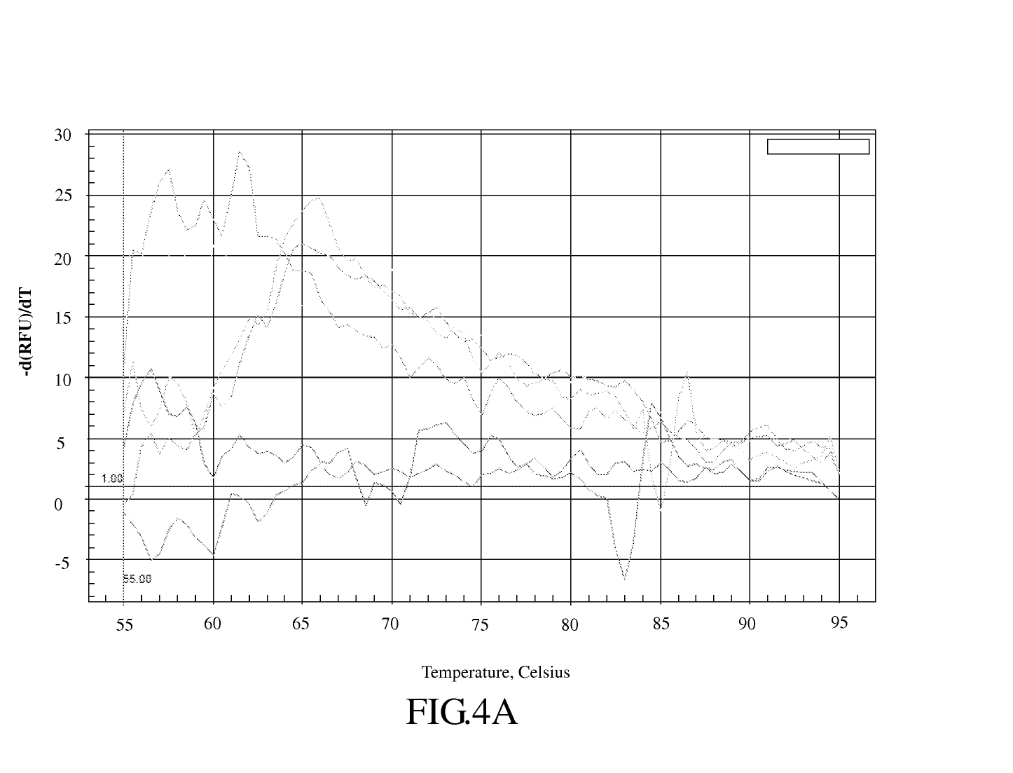

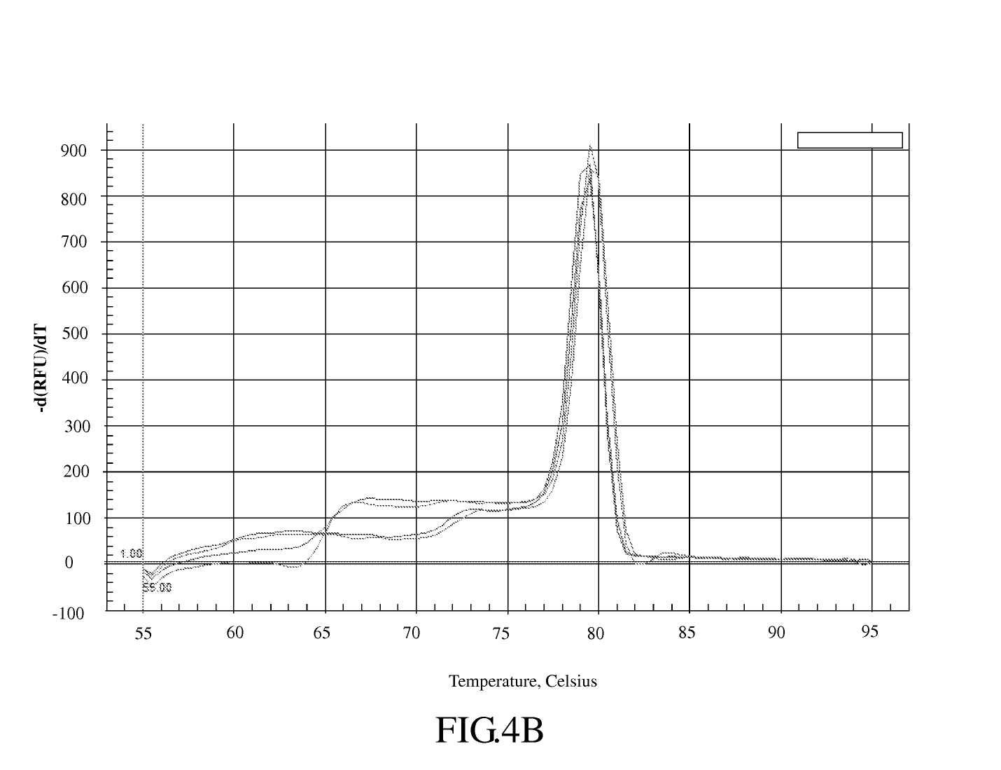

As shown in FIG. 4A, the samples from different subjects are shown with different color lines. There was no specificity PCR product peak, which means that G72 mRNA was not expressed in WBC, and also indicates that the G72 mRNA may be neither a suitable probe for G72 expression level determination nor a suitable probe for diagnosis of schizophrenia in vitro. With reference to FIG. 4B, the GAPDH (housekeeping gene) was used as a control group that expressed only one peak at 80° C.

What is claimed is:

1. A method for diagnosis of schizophrenia, which comprises:
detecting G72 gene product in a body fluid sample from a subject by an assay to determine G72 expression level; comparing said G72 expression level to a baseline G72 expression; and
relating the G72 expression level to the subject's risk of schizophrenia by assigning an increased risk of schizophrenia when said G72 expression level is greater than said baseline G72 expression.

2. The method according to claim 1, wherein the body fluid sample is blood, blood cells, plasma, serum, urine or saliva.

3. The method according to claim 2, wherein the assay is western blotting, enzyme-linked immunosorbent assay, chemiluminescent Immunoassay.

4. The method according to claim 1, which further comprises:
determining G72 expression level of a reference sample from a normal subject to obtain the baseline G72 expression.

5. The method according to claim 2, which further comprises:
determining G72 expression level of a reference sample from a normal subject to obtain the baseline G72 expression.

6. The method according to claim 1, wherein said G72 expression level is at least 2-fold with respect to greater than said baseline G72 expression.

7. The method according to claim 4, wherein said G72 expression level is at least 2-fold with respect to greater than said baseline G72 expression.

8. The method according to claim 5, wherein said G72 expression level is at least 2-fold with respect to greater than said baseline G72 expression.

9. The method according to claim 4, wherein the reference sample is obtained from the normal subject corresponding to the subject in gender or age.

10. A composition for diagnosis of schizophrenia, which comprises: a G72 probe specifically binding to G72 gene product, whereby the G72 probe is adapted to detect expression of G72 gene product; and
a reference sample, which is obtained from a normal subject and provides a baseline G72 expression.

11. The composition for diagnosis of schizophrenia according to claim 10, wherein the G72 gene product is G72 protein or G72 mRNA.

12. The composition for diagnosis of schizophrenia according to claim 10, wherein the G72 probe is anti-G72 antibody.

13. The composition for diagnosis of schizophrenia according to claim 10, wherein the G72 probe contains a polynucleotide with a sequence having at least ten consecutive nucleotides as set forth in SEQ ID NO: 3.

14. The composition for diagnosis of schizophrenia according to claim 10, wherein each primer has at least ten consecutive nucleotides as set forth in SEQ ID NO: 3 to amplify a fragment of the sequence as set forth in SEQ ID NO: 3.

15. The composition for diagnosis of schizophrenia according to claim 10, wherein the primers include polynucleotides respectively having the sequences as set forth in SEQ ID NO: 1 and SEQ ID NO: 2.

16. A method for determination of treatment regimens in subjects of schizophrenia, which includes:
performing an assay that detects G72 gene product in a body fluid sample to determine G72 expression level; comparing said G72 expression level to a baseline G72 expression; and relating the G72 expression level to the subject's risk of schizophrenia by assigning an increased risk of schizophrenia when said G72 expression level is greater than said baseline G72 expression; and
administrating a drug for ameliorating or alleviating symptoms of schizophrenia.
